SEQUENCE LISTING (4 sequences)

SEQ ID NO: 1 - protein, 11 aa, Synthetic construct
DPPPPAVAADV

SEQ ID NO: 2 - protein, 11 aa, Synthetic construct
CRDLLGVVRDF

SEQ ID NO: 3 - protein, 5 aa, Synthetic construct
TYGIQ

SEQ ID NO: 4 - protein, 17 aa, Synthetic construct
WIHAGTGGTKYSRKFQG

(19) United States
(12) Patent Application Publication
(10) Pub. No.: US 2026/0201021 A1
(43) Pub. Date: Jul. 16, 2026

(54) FV-ANTIBODY WITH SPECIFIC BINDING ABILITY TO THE CLEAVAGE SITE OF THE SARS-COV-2 SPIKE PROTEIN BY TMPRSS2

(71) Applicant: UIF (University Industry Foundation), Yonsei University, Seoul (KR)

(72) Inventor: Jae-Chul PYUN, Seoul (KR)

(73) Assignee: UIF (University Industry Foundation), Yonsei University, Seoul (KR)

(21) Appl. No.: 19/225,242

(22) Filed: Jun. 2, 2025

(30) Foreign Application Priority Data
Jan. 13, 2025 (KR) 10-2025-0004851

(51) Int. Cl. C07K 16/10; A61P 31/14

(52) U.S. Cl. CPC C07K 16/104; A61P 31/14; C07K 2317/34; C07K 2317/565; C07K 2317/76; C07K 2317/92

(57) ABSTRACT

Disclosed is a Fv-antibody having a specific binding ability to a cleavage site of a SARS-COV-2 spike protein by TMPRSS2. The Fv-antibody includes a peptide sequence of DPPPPAVAADV or CRDLLGVVRDF.

### CROSS-REFERENCE TO RELATED APPLICATION(S)

This application claims the benefit under 35 USC 119 (a) of Korean Patent Application No. KR 10-2025-0004851 filed on Jan. 13, 2025, with the Korean Intellectual Property Office, the disclosures of which are incorporated herein by reference in their entirety.

INCORPORATION-BY-REFERENCE OF MATERIAL SUBMITTED BY U.S.P.T.O. EFS-WEB

This application contains a Sequence Listing, which is being submitted in computer readable form via the United States Patent and Trademark Office Patent Center and which is hereby incorporated by reference in its entirety for all purposes. The XML file submitted herewith, which is named as “NewApp_1710950013_SequenceListing” and is created on Jun. 2, 2025, contains a 4.58 KB file.

### BACKGROUND

### Field

The present disclosure relates to an Fv-antibody having specific binding ability to a cleavage site of a SARS-COV-2 spike protein by TMPRSS2.

### Description of Related Art

The infection process of severe acute respiratory syndrome coronavirus 2 (SARS-CoV-2) begins with the interaction between spike protein (SP) of SARS-Cov-2 and angiotensin-converting enzyme 2 (ACE2) receptor on host cell. Specifically, the receptor binding domain (RBD) of SP is known to directly interact with the ACE2 receptor. Several strategies have been reported to block this interaction to prevent SARS-COV-2 infection from the attachment to the viral entry of SARS-COV-2 into host cells. However, these strategies have faced challenges due to the frequent mutations occurring at the RBD of SP, which may alter its structure and affect the efficacy of medical interventions.

Consequently, alternative methods have emerged that focus on inhibiting transmembrane protease serine 2 (TMPRSS2) on host cells, a critical enzyme responsible for cleaving the SP during viral entry process. In detail, following the initial binding of the SP to the ACE2 receptor, the membrane fusion between virus and host cells is facilitated by the cleavage of SP. The SP contains cleavage sites located in the proprotein convertase (PPC) region, which includes the S1/S2 site (residues: 682-685) targeted by the proteolytic enzyme furin and the S2′ site (residues: 809-815) targeted by TMPRSS2. This cleavage is essential for the activation of SP, enabling the virus to successfully invade host cells and initiate the infection process. Due to its crucial role in viral entry, TMPRSS2 has gained significant attention as a potential therapeutic target to inhibit SARS-COV-2 infection.

### SUMMARY

A purpose to be achieved by the present disclosure is to develop a new type of a therapeutic agent for effectively inhibiting infection of SARS-COV-2. In particular, the purpose of the present disclosure is to find a novel approach to block the virus's spike protein activation process itself while solving the problem that the conventional RBD-ACE2 binding inhibition approach exhibits limited efficacy as the variants of SARS-COV-2 occur. This novel approach increases a therapeutic possibilities that the approach may cope with a variety of SARS-COV-2 variants, and at the same time may be useful for precisely targeting a virus to host interaction to inhibit the early stages of infection. In addition, the approach may limit the role of TMPRSS2, and thus may be extended to the study of therapeutic agents that may be applied to other coronavirus infections.

In one aspect, the present disclosure provides a peptide capable of specifically binding to a cleavage site of a SARS-COV-2 spike protein by TMPRSS2.

In an embodiment, the peptide may include a peptide sequence of SEQ ID NO: 1 or a peptide sequence of SEQ ID NO: 2:

[SEQ ID NO: 1]

DPPPPAVAADV

[SEQ ID NO: 2]

CRDLLGVVRDF.

In one embodiment, the peptide may specifically bind to a Delta variant among SARS-CoV-2 variants.

In one embodiment, the peptide may specifically bind to Omicron BA.2 and BA.4/5 variants among SARS-COV-2 variants.

In an embodiment, the peptide may bind to a S2′ site of the SARS-COV-2 spike protein to block infection thereof into the host cell.

In one embodiment, the peptide may simultaneously block cleavage of a S1/S2 site and clavate of a S2′ site to prevent an infection process of SARS-COV-2 into the host cell.

In an embodiment, the peptide may bind to an active site (residues D435, S460, and G462) of TMPRSS2 to inhibit cleavage of the spike protein of SARS-COV-2 by the TMPRSS2.

In an embodiment, the peptide may inhibit an interaction between TMPRSS2 in a host cell and SARS-COV-2 spike protein to block exposure of a fusion peptide.

In another aspect, the present disclosure provides a nucleic acid encoding the peptide.

In still another aspect, the present disclosure provides a recombinant expression vector comprising the nucleic acid.

In an embodiment, the recombinant expression vector may include an pET, pGEX, or pCMV-based expression vector.

In an embodiment, the recombinant expression vector may promote expression of a protein neutralizing the SARS-COV-2 spike protein activated by TMPRSS2.

In still another aspect, the present disclosure provides a cell transformed with the recombinant expression vector.

In one embodiment, the cell includes at least one cell selected from the group consisting of animal cells, plant cells, yeast, E. coli, and insect cells.

In one embodiment, the cell includes at least one cell selected from the group consisting of COS7 (monkey kidney cells) cells, NSO cells, SP2/0 cells, CHO (Chinese hamster ovary) cells, W138, BHK (baby hamster kidney) cells, MDCK, myeloma cell lines, HuT 78 cells and HEK293 cells, Escherichia coli, Bacillus subtilis, Streptomyces sp., Pseudomonas sp., Proteus mirabilis or Staphylococcus sp., Aspergillus sp., Pichiapastoris, Saccharomyces cerevisiae, Schizosaccharomyces sp., and Neurosporacrasa.

In one embodiment, the cell may include HEK293 cells in which TMPRSS2 expression is overexpressed.

In one embodiment, the cells may be used in pseudo-virus-based infection experiments of SARS-COV-2.

Still yet another aspect of the present disclosure provides an antibody or antigen-binding fragment thereof that may specifically bind to a cleavage site of a SARS-COV-2 spike protein by TMPRSS2.

In an embodiment, a CDR3 site of the antibody or antigen-binding fragment thereof may include the peptide sequence of SEQ ID NO: 1 or the peptide sequence of SEQ ID NO: 2:

[SEQ ID NO: 1]

DPPPPAVAADV

[SEQ ID NO: 2]

CRDLLGVVRDF.

In one embodiment, the antibody may be a Fv-antibody.

In one embodiment, the Fv-antibody may be produced via an autodisplay system expressed in an outer membrane of E. coli.

In one embodiment, the Fv-antibody may commonly bind to Delta and Omicron variants of SARS-COV-2.

In one embodiment, the CDR3 site of the antibody or antigen-binding fragment thereof may bind to the S2′ site of the SARS-COV-2 spike protein to block infection thereof into the host cell.

In one embodiment, the CDR3 site of the antibody or antigen-binding fragment may bind to H296, D345 and S441 residues contained in an active site of TMPRSS2.

In one embodiment, the antibody may prevent a membrane fusion process between a host cell and the SARS-COV-2 virus.

In still yet another aspect, the present disclosure provides a composition for preventing SARS-COV-2 infection, the composition comprising the antibody or an antigen-binding fragment thereof as described above.

In an embodiment, the composition may further include a pharmacologically acceptable adjuvant for the prevention of SARS-COV-2 infection in addition to the antibody or antigen-binding fragment.

In one embodiment, the composition may be prepared in the form of an eye drop, an injection, or an inhalant.

In one embodiment, the composition may be used not only for the prevention of SARS-CoV-2 infection, but also for treatment after the infection.

Specifically, Fv-antibodies targeting the transmembrane protease serine 2 (TMPRSS2) were screened from an Fv-antibody library to inhibit SARS-COV-2 infection. Fv-antibodies indicated the variable region of heavy chain immunoglobulin G (IgG) with three complementarity-determining regions (CDRs) and frame regions (FRs), and Fv-antibody library was prepared through the site-directed mutagenesis of CDR3. The proteolytic cleavage site (S2′ site) of TMPRSS2 on spike protein of SARS-COV-2 was used as a screening probe of Fv-antibody library. Two Fv-antibodies were screened and subsequently expressed as soluble recombinant proteins. The binding affinities of expressed Fv-antibodies were estimated using a surface plasmon resonance (SPR) biosensor. Two expressed Fv-antibodies were analyzed to bind specifically at the cleavage site of TMPRSS2 (S2′ site) using a SPR biosensor. The neutralizing activities of two expressed Fv-antibodies were demonstrated using a cell-based infection assay with pseudo-viruses which expressed the spike proteins (SP) of four kinds of SARS-COV-2 variants, such as Wu-1 (D614), Delta (B.1.617.2), Omicron (BA.2), and Omicron (BA.4/5). Additionally, the docking simulation was carried out to analyze the interaction of screened Fv-antibodies and the active sites of TMPRSS2.

The effect of the present disclosure is that the subject matters including the peptide or the antibody or antigen-binding fragment thereof of the present disclosure increase the coping ability with various viral strains, including variants, by blocking the infection process of SARS-CoV-2 early. In particular, the subject matters including the peptide or the antibody or antigen-binding fragment thereof of the present disclosure may simultaneously target the active site of TMPRSS2 and the S2′ cleavage site of the spike protein to maximize the potential of the infection inhibition. This mechanism provides the potential to exert stable inhibitory effects on the variant even if the structure of the spike protein is changed into the variant. This complements the limitations of existing treatments and has the potential to be used not only for infection prevention but also for early infection treatment.

Effects of the present disclosure are not limited to the effects mentioned above, and other effects not mentioned will be clearly understood by those skilled in the art from the description as set forth below.

In addition to the above effects, specific effects of the present disclosure are described together while describing specific details for carrying out the present disclosure.

### BRIEF DESCRIPTION OF DRAWINGS

FIG. 1 is a diagram illustrating the prevention of SARS-COV-2 infection using a TMPRSS2 inhibitor screened from the Fv-antibody library.

FIG. 2A is a diagram showing a procedure for generating various antibodies via a Fv-antibody library production process and randomization of the CDR3 region and screening the various antibodies and evaluating antigen-binding properties of the screened antibodies.

FIG. 2B is a diagram showing a structural domain of a TMPRSS2 protein, an active site thereof, and a probe fabrication process for detecting the same.

FIG. 2C is a diagram illustrating a process of screening antibodies that bind to TMPRSS2 using cells having expressed randomized CDR3 in the Fv-antibody library.

FIG. 2D is a diagram showing a process using flow cytometry (FACS) to screen the antibodies that bind to the TMPRSS2 probe in a Fv-antibody library.

FIG. 2E is a diagram showing the analysis results of the screened Fv-antibody clone and the CDR3 sequence of the clone.

FIG. 3A is a diagram illustrating a recombination process of each of screened Fv antibodies including the CDR3.

FIG. 3B is a diagram showing the results of analyzing the binding affinity (K_{D}) of each of Anti-TMPRSS2 Fv-1 and Fv-2 antibodies.

FIG. 4A is a view showing the results of identifying the process of inhibiting the interaction between the S2′ site of SARS-COV-2 spike protein and TMPRSS2 using the Fv-antibody, based on SPR analysis.

FIG. 4B is a diagram showing the results of identifying the interaction between the S2′ site of the SARS-COV-2 spike protein and TMPRSS2, based on SPR analysis.

FIG. 4C is a diagram showing the results of analyzing the effect of inhibiting the interaction between TMPRSS2 treated with the Fv antibodies (Fv-1 and Fv-2) and the S2′ site of SARS-COV-2 spike protein, based on IC50 values.

FIG. 5A is a diagram showing the structure of the TMPRSS2 protein and the position thereof at which the Anti-TMPRSS2 Fv-1 antibody binds thereto.

FIG. 5B is a diagram showing the structure of the TMPRSS2 protein and the position thereof at which the Anti-TMPRSS2 Fv-2 antibody binds thereto.

FIG. 5C shows the amino acids of TMPRSS2 via which the TMPRSS2 interacts with Anti-TMPRSS2 Fv-1 and Fv-2 antibodies, and binding energies thereof.

FIG. 6A shows a process of inhibiting infection of SARS-COV-2 variants (Wu-1, Delta, and Omicron) into the host cell by blocking S2′ site cleavage of TMPRSS2 using the Anti-TMPRSS2 antibody.

FIG. 6B is a diagram showing the fluorescence microscopy measurement result of identifying the binding of Anti-TMPRSS2 Fv-1 and Fv-2 antibodies to the TMPRSS2 in the cell in a state in which Anti-TMPRSS2 Fv-1 and Fv-2 antibodies are labeled with GFP.

FIG. 6C is a diagram showing the fluorescence microscopy observation result of observing the SARS-COV-2 infection into the cells treated with Anti-TMPRSS2 Fv-1 and Fv-2 antibodies.

FIG. 6D is a diagram showing the normalized infection ratio indicating the neutralizing effect of each of the Anti-TMPRSS2 Fv-1 and Fv-2 antibodies on the Wu-1, Delta, Omicron BA.2, and Omicron BA.4/5 variants of SARS-COV-2.

### DETAILED DESCRIPTIONS

Advantages and features of the present disclosure, and a method of achieving the advantages and features will become apparent with reference to embodiments described later in detail together with the accompanying drawings. However, the present disclosure is not limited to the embodiments as disclosed under, but may be implemented in various different forms. Thus, these embodiments are set forth only to make the present disclosure complete, and to completely inform the scope of the present disclosure to those of ordinary skill in the technical field to which the present disclosure belongs, and the present disclosure is only defined by the scope of the claims.

Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure. Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “comprising”, “include”, and “including” when used in the present disclosure, specify the presence of the stated features, integers, operations, elements, and/or components, but do not preclude the presence or addition of one or more other features, integers, operations, elements, components, and/or portions thereof. As used herein, the term “and/or” includes any and all combinations of one or more of associated listed items. Expression such as “at least one of” when preceding a list of elements may modify the entire list of elements and may not modify the individual elements of the list. In interpretation of numerical values, an error or tolerance therein may occur even when there is no explicit description thereof.

In interpreting a numerical value, the value is interpreted as including an error range unless there is no separate explicit description thereof. In the context of the present disclosure, the term “about” may mean about ±1%, about ±2%, about ±3%, about ±4%, about ±5%, about ±6%, about ±7%, about ±8%, about ±9%, or about ±10% of a value stated herein.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

A peptide according to an embodiment of the present disclosure may specifically bind to a cleavage site of SARS-COV-2 spike protein by TMPRSS2. When the peptide specifically binds to the cleavage site of the SARS-COV-2 spike protein by TMPRSS2, the TMPRSS2 is unable to efficiently cleave the S2′ site of the spike protein, thereby inhibiting the exposure of the fusion peptide thereto. This hinders the fusion process between the virus and the host cell membrane, making it difficult for the virus to invade the host cell. Ultimately, this mechanism may contribute to the inhibition of replication and spread of SARS-COV-2.

In the context of the present disclosure, the term SARS-COV-2 refers to severe acute respiratory syndrome (SARS) coronavirus-2 and is an RNA virus that infects host cells, including humans, to cause COVID-19. The virus uses spike proteins to interact with host cells to cause the infection.

In the context of the present disclosure, the term spike protein refers to a protein in the form of a protrusion expressed on the surface of SARS-COV-2, which plays a major role in mediating the invasion of the virus into the cell by interacting with the ACE2 receptor and TMPRSS2 of the host cell.

In the context of the present disclosure, the term TMPRSS2 refers to a serine protease present in the host cell membrane and refers to an enzyme that cleaves the S1/S2 and S2′ sites of a spike protein to activate a fusion peptide to promote fusion of the virus with the host cell membrane.

In the context herein, in the context of a virus and a host, the meaning of the term the fusion refers to the process in which a spike protein of the virus interacts with the host cell membrane, such that the two membranes bind to each other and integrate into a single membrane, whereby the viral genome is transferred into the host cell.

In the context of the present disclosure, the term the fusion peptide refers to a specific amino acid sequence in the S2 region of a SARS-COV-2 spike protein that is activated by the TMPRSS2 and exposed to promote physical fusion of the virus with a host cell membrane.

In one embodiment, the peptide may include a peptide sequence of DPPPPAVAADV or CRDLLGVVRDF.

In one embodiment, the peptide may specifically bind to a Delta variant among SARS-CoV-2 variants. In one embodiment, the peptide may specifically bind to Omicron BA.2 and BA.4/5 variants among SARS-COV-2 variants. In the context of the present disclosure, the term SARS-COV-2 variant refers to a modified virus strain in which a mutation occurs in the gene sequence of the SARS-COV-2 virus to exhibit a structural or functional difference from an existing virus.

In the variant, the specific amino acid sequence of the spike protein may be modified or the structural properties of the spike protein may be changed such that infectivity, propagation power, vaccine avoidance ability, or antibody reactivity may be different from those of the non-variant. The delta variant has a specific mutation including P681R mutation in the spike protein and is characterized by high propagation power. It is known that the BA.2 and BA.4/5 sublineages of Omicron variants contain a number of mutations in the spike protein, and have a relatively stronger immune evasion ability compared to conventional variants. The term SARS-COV-2 variants used herein encompasses all strains with these characteristics.

The types of SARS-COV-2 variants are classified according to mutations that occur in gene sequences including the spike protein, and different variants exhibit different characteristics in the infectivity, propagation power, and immune evasion ability. The major variants include the Alpha variant (B.1.1.7), the Beta variant (B.1.351), the Gamma variant (P.1), the Delta variant (B.1.617.2), and the Omicron variant (B.1.1.529). In particular, Omicron variants are sub-classified into sublineages such as BA.1, BA.2, BA.4, BA.5, and XBB, which have a number of mutations to exhibit enhanced ability to evade vaccines and natural immunity. In addition, the variants having various regional or clinical characteristics have been reported, and World Health Organization (WHO) classifies the variants into Variants of Concern (VOC), Variants of Interest (VOI), or Variants under Monitoring (VUM) and monitors the variants in the classified manner. These variants have been continuously generated and reported as the virus evolves and spreads.

The peptide according to the present disclosure may prevent the host cell from being infected with the SARS-COV-2 variant without being significantly affected by the type of the SARS-COV-2 variant. This is because the peptide according to the present disclosure specifically binds to the cleavage site of the SARS-COV-2 spike protein by TMPRSS2, which is less likely to be mutated, especially the S2′ site. The S2′ site is an essential site that must be activated for the virus to invade the host cell, and this site tends to be conserved structurally among variants of the virus.

Therefore, unlike the conventional therapeutic agent that targets the site where the mutation occurs frequently, such as the receptor binding domain (RBD) of the spike protein, the peptide according to the present disclosure may act stably without being significantly affected by the characteristics of the variant. In addition, the TMPRSS2 is a host cell protein which is not affected by the mutation. Thus, the peptide according to the present disclosure may provide an effective infection blocking mechanism even against various variants, and is highly likely to exert an infection blocking effect under the same mechanism without being affected by structural changes in the spike protein of the variants that will occur in the future.

In an embodiment, the peptide may bind to the S2′ site of the SARS-COV-2 spike protein to block infection of the virus into the host cell. In the context of the present disclosure, the term the S2′ site refers to a specific cleavage site which is located within the S2 region of the SARS-COV-2 spike protein (SP) and which is cleaved by TMPRSS2 and plays a key role in promoting fusion of the virus with the host cell membrane. The S2′ site is composed of amino acid residues 809 to 815 in the pro-peptide region of the spike protein. Only when the cleavage of this site occurs, the fusion peptide may be exposed to enable physical binding of the virus and the host cell membrane to each other. The S2′ site has high conservation among various SARS-COV-2 variants due to the structural and functional nature of the spike protein. This is one of the main reasons why the peptide according to the present disclosure may target this site to exert the infection blocking effect.

In one embodiment, the peptide may simultaneously block cleavage of the S1/S2 site and the S2′ site to prevent the infection process of SARS-COV-2 in the host cell. In this case, activation of the spike protein is inhibited in duplicate, making it impossible for the virus to proceed with the membrane fusion process even if it binds to the host cell. Cleavage of the S1/S2 site inhibits the separation between the S1 and S2 regions of the spike protein, rendering the fusion peptide nonfunctional, and cleavage of the S2′ site prevents exposure of the fusion peptide, making physical fusion between the virus and the host cell membrane impossible.

In an embodiment, the peptide may bind to an active site (D435, S460, and G462 residues) of TMPRSS2 to inhibit cleavage of the spike protein of SARS-COV-2 by TMPRSS2. This mechanism prevents the TMPRSS2 from efficiently cleaving the S1/S2 site and the S2′ site of the spike protein, thereby inhibiting the activation of the fusion peptide essential for host cell invasion of the virus. Consequently, the structural modification of the spike protein and the membrane fusion process are blocked, thereby preventing the entry of the virus into the cells.

Each of the D435, S460 and G462 residues represents a specific amino acid position within the active site of the TMPRSS2 protein and play an important role in the cleavage process of the SARS-COV-2 spike protein. D435 is aspartic acid, which is negatively charged and contributes to binding and stabilization with the substrate, that is, the spike protein. S460 is serine, and is a key residue constituting the catalytic triad of TMPRSS2, and plays a role in directly mediating the cleavage reaction of the spike protein. G462 is glycine, which has a high structural flexibility and constitutes a substrate binding pocket at the TMPRSS2 active site, thereby securing the spike protein. These residues are essential for determining the enzymatic activity and substrate specificity of TMPRSS2. Thus, the peptide according to the present disclosure may bind to these residues to inhibit the cleavage action of TMPRSS2 and prevent the host cell from being infected with the virus.

In an embodiment, the peptide according to the present disclosure may inhibit an interaction between TMPRSS2 in the host cell and SARS-COV-2 spike protein, thereby blocking exposure of the fusion peptide. Blocking the exposure of the fusion peptides may allow the process in which the SARS-COV-2 spike protein interacts with the host cell membrane to induce membrane fusion to be suppressed. This prevents the virus from fusing with the cell membrane of the host cell, thus preventing the viral genome from being transferred to the host cytoplasm. Consequently, the replication cycle of the virus does not begin and is inhibited at the early stages of infection. Such blocking may contribute to reducing the spread of the virus and preventing damage to host cells, and the risk of infection by variants may also be effectively mitigated. Thus, the peptide according to the present disclosure provides the possibility to be utilized for the prevention and treatment of SARS-COV-2 and pseudo-viral infections.

In one example, the nucleic acid according to an embodiment of the present disclosure may encode the peptide. In the context of the present disclosure, the dictionary meaning of a nucleic acid is a biochemical substance that stores and conveys genetic information, and includes DNA or RNA. These nucleic acids are components of genes and contain information necessary for the biosynthesis of proteins and peptides. In the context of the present disclosure, the dictionary meaning of the nucleic acid encoding the peptide means that the nucleic acid (DNA or RNA) has genetic information that instructs the synthesis of a peptide composed of a specific amino acid sequence. This information is converted into the peptide via transcription and translation processes. During the transcription process, the genetic information of DNA is copied into mRNA, and during the translation process, the code of mRNA is translated into an amino acid sequence by the ribosome, resulting in the synthesis of the peptide. Through this process, the nucleic acid directly plays a role in determining the structure and function of the peptide.

As known in the art, a combination of nucleic acids encoding the amino acid included in the peptide may vary. Accordingly, according to the present disclosure, there are proposed not only the above-described peptide but also a nucleic acid encoding the above-described peptide, wherein the nucleic acid includes all of theoretical 1,048,576 nucleic acid sequences encoding the SEQ ID NO: 1 and theoretical 1,327,104 nucleic acid sequences encoding the SEQ ID NO: 2. These diverse nucleic acid sequences are attributed to the codon variability of the amino acids. A codon is three consecutive bases of the nucleic acid encoding one amino acid, and multiple codons may encode the same amino acid, so that various nucleic acid sequences for encoding the same peptide sequence may be present. This increases the flexibility of the subject matter of the present disclosure and may help to select nucleic acid sequences optimized for specific biological systems or applications. For example, the nucleic acid sequences may be optimized by taking into account use of the codon that is translated more efficiently in certain species of organisms. This may contribute to enhancing the expression level and stability of the peptide.

In one example, a recombinant expression vector according to an embodiment of the present disclosure may include the nucleic acid. In the context of the present disclosure, the dictionary meaning of the recombinant expression vector is a molecule used to introduce a gene into another cell to express a protein or a peptide. This vector may take the form of plasmids, viruses, artificial chromosomes, etc., and includes essential elements for replication and gene expression, such as selection markers, promoters, reporter genes, etc.

Since the recombinant expression vector includes the nucleic acid, the nucleic acid may be expressed in a host cell to produce a target product such as a peptide or an antibody fragment. Thus, a functional protein that blocks the interaction between TMPRSS2 and SARS-CoV-2 spike proteins is effectively generated.

The type of the recombinant expression vector is not particularly limited. In one embodiment, the recombinant expression vector may include the following series of expression vectors. The pET vector is mainly used in bacterial expression systems and contains a strong T7 promoter that enables high-efficiency protein expression, thereby enabling the mass production of a target protein in host cells such as E. coli. The pGEX vector enables easy purification of the expressed protein using a Glutathione S-Transferase (GST) tag and may purify the protein at a high purity level via affinity chromatography of the protein after cell lysis. The pCMV vector is suitable for eukaryotic cell expression systems and enables high levels of protein expression in hosts such as human cells via a strong CMV (Cytomegalovirus) promoter.

Examples of non-limiting recombinant expression vectors may include various vectors such as pET-28a, pET-21d, pGEX-4T-1, pGEX-6P-1, pCMV-Tag2B, and pcDNA3.1. These vectors are designed to be adapted to a variety of host systems, such as bacteria, eukaryotic cells, and insect cells, thereby facilitating the expression and purification of the target product. The recombinant expression vectors used in the present disclosure may be selectively used in combination with each other according to the characteristics of an expression system and a target protein.

In the context of the present disclosure, the term the neutralization means interfering with or blocking a process in which a virus such as SARS-COV-2 invades the host cell, thereby causing an infection. This is accomplished by inhibiting the process in which viral spike proteins interact with receptors or enzymes in the host cell membrane or by blocking physical fusion between the host cell and the virus. The neutralization is an important mechanism that may inhibit viral replication and contribute to effectively blocking infection.

In the context of the present disclosure, the term the expression of the protein refers to a process in which a nucleic acid sequence included in the recombinant expression vector is produced into a specific protein via transcription and translation processes in a host cell. This involves a series of processes in which the nucleic acid sequence is transcribed into mRNA, and the mRNA is translated by ribosomes to synthesize the target protein. This process may be regulated according to the promoter of the recombinant expression vector, the expression system of the host cell, and environmental conditions.

In one example, a cell according to an embodiment of the present disclosure may be transformed with the recombinant expression vector. In the context of the present disclosure, the dictionary meaning of the transformation of the cell is a process of changing the genetic composition of the cell by introducing the DNA from the outside thereto. Through this process, the cell acquires a new gene, which may be expressed in the cell to produce a specific protein.

As the cells are transformed with the recombinant expression vector, continuous production of a specific peptide or protein may be possible. This process allows the cell to receive the genetic information and perform new biological functions based on the genetic information. In the present disclosure, the transformed cell may be utilized for the production of proteins or peptides that inhibit infection of SARS-COV-2 or block interaction between viruses and host cells. Thus, the transformed cell acts as a system capable of mass-producing a biological agent having a virus neutralizing effect, and has the possibility of being applied not only to the development of therapeutic agents and prophylactic agents but also to infection research models.

In an embodiment, the cell may include one or more cells selected from the group consisting of animal cells, plant cells, yeast, E. coli, and insect cells. In one embodiment, the cells may include one or more cells selected from the group including COS7 (monkey kidney cells) cells, NSO cells, SP2/0 cells, CHO (Chinese hamster ovary) cells, W138, BHK (baby hamster kidney) cells, MDCK, myeloma cell lines, HuT 78 cells and HEK293 cells, Escherichia coli, Bacillus subtilis, Streptomyces sp., Pseudomonas sp., Proteus mirabilis or Staphylococcus sp., Aspergillus sp., Pichiapastoris, Saccharomyces cerevisiae, Schizosaccharomyces sp., and Neurosporacrasa.

According to the present disclosure, using the various cells as described above, a system for producing a target peptide or protein via the recombinant expression vector may be flexibly implemented. The level of protein expression, post-translation modification, and easiness of purification vary depending on the cell type. Thus, according to the present disclosure, an optimal expression system may be selected and utilized according to the characteristics and use of the target protein. For example, animal cells may provide post-translation modifications similar to that in the human proteins, and may be suitable for therapeutic protein production. The bacterial cells such as E. coli are advantageous for mass production of proteins and rapid culture thereof. In addition, yeast and insect cells provide a high-efficiency expression system and a relatively simple culture process, and may be suitable for industrial mass production.

In one embodiment, the cell may include HEK293 cells in which TMPRSS is overexpressed. The HEK293 cells are cell lines derived from human embryonic kidney cells and are genetically modified so as to have high growth rates and stable protein expression characteristics. Overexpression of TMPRSS2 refers to a state in which TMPRSS2 protein is expressed at a level much higher than the normal level in the cells using recombinant technology or a specific promoter.

In one embodiment, the cells may be used in the pseudo-virus of SARS-COV-2 based infection experiments. The pseudo-virus refers to an inactive virus that has been experimentally produced by mimicking some structural or genetic characteristics of the authentic virus. In the pseudo-virus, pathogenicity is eliminated or attenuated such the pseudo-virus is commonly used to study the mechanism of infection of viruses or to evaluate the efficacy of vaccines, antibodies, drugs, etc.,. The pseudo-virus is designed such that the pseudo-virus contains a key target structure, such as the spike protein of the target virus, while the viral genome is modified so as not to cause pathogenicity or the pseudo-virus is non-replicable.

The pseudo-virus of SARS-COV-2 is a virus genetically engineered to express spike proteins, and may reproduce the process of binding and infection with host cells. The use of the pseudo-virus allows safe construction of SARS-COV-2 infection models without the need for direct handling of high-risk pathogens. The pseudo-virus may be exploited for a variety of experimental purposes, such as drug screening, antibody neutralization experiments, and virus-host interaction studies.

In one example, an antibody or antigen-binding fragment thereof according to an embodiment of the present disclosure may specifically bind to a cleavage site of SARS-CoV-2 spike protein by TMPRSS2. In one embodiment, the CDR3 site of the antibody or antigen-binding fragment thereof may comprise a peptide sequence of DPPPPAVAADV or a peptide sequence of CRDLLGVVRDF. In the context of the present disclosure, the term CDR3 refers to a third region of the Complementarity-Determining Region (CDR) that determines the binding specificity with an antigen in the variable region of an antibody. The CDR3 is usually the most variable region within the variable regions of the VH (heavy chain) and VL (light chain) of the antibody, and plays a key role in forming a unique binding to a specific epitope of an antigen.

In one embodiment, the antibody may be a Fv-antibody. In the context of the present disclosure, the meaning of the Fv-antibody refers to the smallest unit that maintains the antigen-binding function of the antibody and refers to a fragment composed of of VH (variable region of heavy chain) and VL (variable region of light chain). The Fv-antibody is smaller in size than the traditional complete antibody and thus easily penetrates into cells. The Fv-antibody contains an antigen-binding site, that is, the CDR (Complementarity-Determining Region), and thus maintains the antigen specificity. In addition, the Fv-antibody may be efficiently expressed in bacteria, yeast, animal cells, etc. via various recombinant technologies, and thus may be usefully used for research and treatment purposes. In an embodiment of the present disclosure, the Fv-antibody specifically binds to the cleavage site of the SARS-COV-2 spike protein by the TMPRSS2, thereby providing the possibility of inhibiting the viral infection process.

In one embodiment, the Fv-antibody may be produced via an autodisplay system expressed in the outer membrane of E. coli. In the context of the present disclosure, the dictionary meaning of E. coli is Escherichia coli, and refers to one type of gram-negative bacterium. In the context herein, the meaning of the Autodisplay refers to a technique for expressing a recombinant protein in the outer membrane of a bacterium such as E. coli.

In one embodiment, the Fv-antibody can commonly bind to Delta and Omicron variants of SARS-COV-2. Non-limiting examples of other variants of SARS-COV-2 to which the Fv-antibody is capable of binding may include Alpha variants (B.1.1.7), Beta variants (B.1.351), Gamma variants (P.1), Lambda variants (C.37), Mu variants (B.1.621), and new variants occurring in the future. These variants include mutations in the receptor binding domain (RBD) or the N-terminal domain (NTD) of the spike protein. In this regard, the Fv antibodies of the present disclosure may target the S2′ site which has low mutations and high conservation ability and thus maintain binding potential to various variants.

In one embodiment, the CDR3 site of the antibody or antigen-binding fragment thereof may bind to the S2′ site of the SARS-COV-2 spike protein to block infection of the virus into the host cell. This suppresses the process of cleavage of the S2′ site by TMPRSS2, thereby inhibiting the exposure of the fusion peptide and blocking the physical fusion between the virus and the host cell membrane. Consequently, the virus is unable to transfer the genome into the host cell, thereby inhibiting the infection. This acts as a common mechanism among the various variants, thereby providing the possibility of effectively blocking the early stages of SARS-COV-2 infection.

In one embodiment, the CDR3 site of the antibody or antigen-binding fragment may bind to H296, D345 and S441 residues contained in the active site of TMPRSS2. This prevents the catalytic triad of TMPRSS2 from cleaving the S1/S2 and S2′ sites of the spike protein, thereby inhibiting the activation process of the spike protein. Consequently, the exposure of the fusion peptide is blocked, such that the virus is unable to physically bind to the host cell. This prevents the invasion of the virus into the cell to provide the possibility to block the transmission of SARS-COV-2 in the early stages of infection.

In one embodiment, the antibody may prevent a membrane fusion process between a host cell and the SARS-COV-2 virus. Thus, the physical fusion between the virus and the host cell membrane is prevented, and thus, the viral genome is not transferred to the host cytoplasm. This fundamentally inhibits the replication and proliferation of the virus, and prevents the production of new viral particles in infected host cells to effectively block the spread of the infection. This mechanism may contribute to reducing damage to host cells and mitigating infection-related complications.

In one example, a composition for preventing SARS-COV-2 infection according to an embodiment of the present disclosure may include the antibody or antigen-binding fragment thereof as described above. The composition may inhibit the cleavage of the SARS-COV-2 spike protein by TMPRSS2 to block the process in which the virus invades the host cell. The antibody or antigen-binding fragment thereof contained in the composition may bind to highly conservative viral sites, such as the S2′ site, thereby providing a consistent preventive effect against a variety of variants. In addition, the composition may be applied to various formulations such as eye drops, sprays, injections, or inhalants, and thus has the potential to improve convenience and efficiency in preventing the infection. The composition may inhibit the spread of SARS-COV-2 and its variants and provide prophylactic protection in high-risk environments.

In an embodiment, the composition may further include a pharmacologically acceptable adjuvant for the prevention of SARS-COV-2 infection in addition to the antibody or antigen-binding fragment. Non-limiting examples of the adjuvants are as follows. Aluminum salts (aluminum hydroxide, aluminum phosphate) that may be used to enhance the immune response or to increase the stability of the composition, immune adjuvants such as AS01, AS03, MF59, lipid-based adjuvants such as squalene, cholesterol, Monophosphoryl Lipid A (MPLA), TLR agonists such as CpG oligonucleotides or poly I: C, antibody adjuvants such as Imiquimod, QS-21, immunoglobulin G (IgG) and M (IgM), viscosity modulators such as hyaluronic acid or carboxymethylcellulose, osmotic modulators such as saline and phosphate buffer (PBS), stabilizers such as glycerol, mannitol, sorbitol, protein stabilizer such as human serum albumin (HBS), preservatives such as trehalose, dextran, polysorbate 20 or 80, nanomaterial-based formulations such as cyclodextrin, ethylenediaminetetraacetic acid (EDTA), gas preservatives such as carbon dioxide and nitrogen, biocompatible substances such as propylene glycol, PEG (polyethylene glycol), polylactic coglycolyacid (PLGA), antimicrobial adjuvants such as benzalkonium chloride, chlorhexidine, antioxidants such as N-acetylcysteine, tocopherol, glutathione, surfactants such as cetylpyridinium chloride, pH modifiers such as heptadixestran, alginic acid, formic acid, sodium citrate, natural extracts such as eucalyptus extract, elderberry extract, vitamins and minerals such as calcium nitrate, sodium ascorbate, skeletal reinforcing aids such as clodronate, bisphosphonate, cooling agents such as menthol, camphor, applicable protein and lipid-based adjuvants such as vegetable oils, protein-based materials such as casein, porous materials such as zeolite, microcrystalline cellulose, nanoparticle-based materials, gelatin, glycine, lecithin. Such adjuvants may be used to adjust the physical, chemical, and biological properties of the composition to enhance stability, efficacy, and applicability thereof.

In one embodiment, the composition may be prepared in the form of an eye drop, an injection, or an inhalant. Other non-limiting examples of formulations for applying the composition to a living body may include oral formulations, nasal sprays, transdermal patches, suppositories, sustained release tablets, oral films, nanoparticle-based drug delivery systems, liposome formulations, microbeads, hydrogels, suspensions, emulsions, ointments, creams, lotions, deformable nanogels, implantable devices, biodegradable microneedles, mucosal absorption patches, powder inhalers, transdermal infusion systems, water-soluble polymer formulations, particulate-based transporters, lyophilized formulations for vaccination, biopolymer-based microstructures, osmotic pressure control tablets, solutions in mist form, liquefied gas-based transporters, ultrasound-induced drug delivery, bioadaptive films, metered dose inhalers (MDIs), soft capsules, solid powder disks, multiple capsular tablets, intraoral sprays, implantable pump systems, suspension injectable agents, microgel particles for in vivo injection, bioadaptive nanofibers, implantable devices, bioprinting-based intra-tissue transporters, biodegradable influencer particles, etc. These formulations may be designed to be adapted to various bio-application pathways, thereby improving the efficacy, stability, and delivery efficiency of the composition.

In one embodiment, the composition may be used not only for the prevention of SARS-COV-2 infection, but also for treatment after the infection. The reason why the composition may have a therapeutic effect is that it includes a mechanism capable of inhibiting the spread of the virus and cell damage not only in the early stages of SARS-COV-2 infection but also in the advanced state of infection. The antibody or antigen-binding fragment contained in the present composition specifically binds to a cleavage site of the SARS-COV-2 spike protein by TMPRSS2 or the S2′ site thereof, thereby blocking the exposure of the fusion peptide and fusion thereof with the host cell membrane. This provides the effect of inhibiting the formation of new viral particles in infected cells and preventing further infection into adjacent healthy cells.

In addition, the antibody included in the composition may neutralize viral particles or interfering with interaction of the virus with host cells, and thus may have the potential to enhance the immune response and promote the removal of infected cells. Immunomodulators or anti-inflammatory drugs that may be contained in the composition as the adjuvants may help relieve excessive inflammatory reactions and restore the balance of the immune system to reduce tissue damage caused by the infection. Due to this complex action, the composition has the potential to be utilized not only for the prevention of SARS-COV-2 infection but also for therapeutic purposes thereof. n

An advantage of the peptide, the antibody or antigen-binding fragment thereof including the same, and the composition for prevention and/or treatment including the same according to an embodiment of the present disclosure is that the peptide specifically binds to a structurally conserved region such as a cleavage site of a SARS-COV-2 spike protein by TMPRSS2, thereby exhibiting stable effects on various variants. These peptides and antibodies block infection by inhibiting the early stages of the interaction between the virus and the host cell, providing the potential to slow or mitigate the progression of the disease after infection. The composition may be prepared in various formulations such as eye drops, injections, inhalants, etc., thereby enhancing the convenience of use thereof and accessibility thereto, and may be used not only for preventing infection but also for therapeutic purposes. In addition, the adjuvants are included in the composition so as to provide complex functions such as immune response control, inflammation relief, and increased therapeutic effect, thereby supplementing the limitations of a single mechanism therapy. With these characteristics, the compositions of the present disclosure have high efficacy and applicability in the prevention and treatment of SARS-COV-2 and variant infections.

Hereinafter, examples of the present disclosure will be described. However, the examples as described below are only some implementations of the present disclosure, and the scope of the present disclosure is not limited to the following examples.

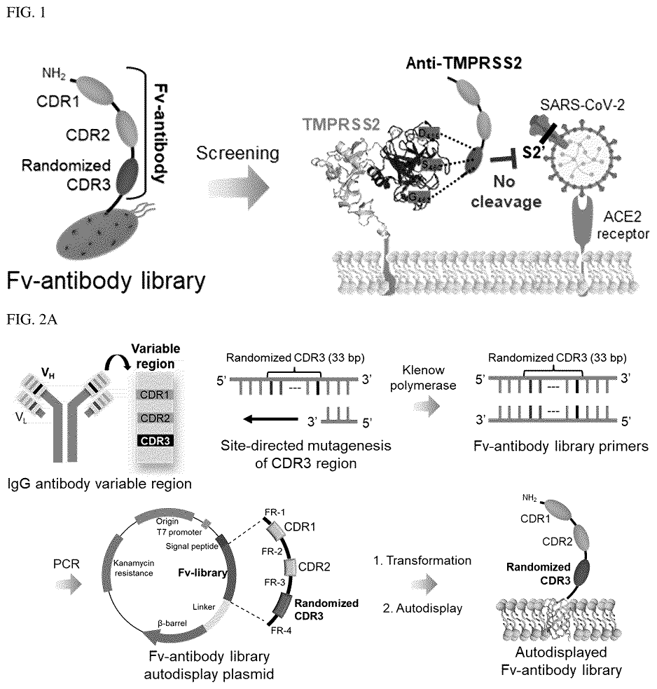

In the present disclosure, the Fv-antibodies targeting the TMPRSS2 were screened from an Fv-antibody library to inhibit SARS-COV-2 infection. As shown in FIG. 1, Fv-antibodies, which are derived from the variable heavy (VH) region of heavy chain immunoglobulin G (IgG), consists of three complementarity-determining regions (CDRs) and four framework regions (FRs). The CDR3 region of Fv-antibodies was randomized through site-directed mutagenesis. So prepared Fv-antibody library was expressed on the surface of E. coli using autodisplay technology, which utilizes the AIDA-1 system to transport the target proteins to the bacterial surface. The library showed a high display density, with 10⁵ Fv-antibodies per E. coli, and exhibited a diversity of 10⁶ different Fv-antibodies per library.

Fv-antibodies were screened using extracellular domain of TMPRSS2 (residue: 106-492), including the active sites (residues: 256-489), substrate binding site (residues: D435, S460 and G462), and catalytically active site (residues: H296, D345 and S441) as a screening probe. This region was located in the pocket where S2′ site of SP was known to specifically bind, facilitating the proteolytic cleavage necessary for viral entry. The screened Fv-antibodies were expressed as soluble recombinant proteins, and the specific binding activity to the TMPRSS2 was analyzed using a surface plasmon resonance (SPR) biosensor.

In order to demonstrate the inhibition of SARS-COV-2 infection using the screened Fv-antibodies, the neutralizing activity of these Fv-antibodies was carried out using cell-based infection assay with pseudo-virus particles expressing the SPs of different SARS-CoV-2 variants, including Wu-1 (D614), Delta (B.1.617.2), Omicron (BA.2), and Omicron (BA.4/5), respectively. Additionally, the docking simulation was carried out to analyze the interaction of screened Fv-antibodies and the active sites of TMPRSS2.

### Materials and Methods

### [Materials]

The expression vector for TMPRSS2 (residues: 106-492, 69.7 kDa) and the SARS-COV-2 PPC (residues: 661-900, 53.6 kDa) were synthesized by Cosmo Genetech (Seoul, South Korea). The materials necessary for producing SARS-COV-2 variants pseudo-virus were described in a previous study. Pseudotyped SP variants vectors and HEK-Blue™ cells, which overexpress hACE2-TMPRSS2, were obtained from InvivoGen Inc. (San Diego, CA, USA). Dulbecco's modified Eagle's medium (DMEM) and Opti-MEM were sourced from Gibco Inc. (Waltham, MA, USA).

### [Screening of Anti-TMPRSS2 Fv-Antibodies]

The preparation of the Fv-antibody library was described in previous studies. Clones exhibiting binding activity to the TMPRSS2 probe (extracellular residue: 106-492, labeled with GFP) were screened using the following protocol: (1) The randomized CDR3 region of the Fv-antibody was displayed on the surface of E. coli. (2) The TMPRSS2 probe (1 μM, 100 μL) was incubated with the Fv-antibody library (100 μL) for 1 h at 37° C. (3) After washing with 0.01% PBST (containing 0.01% Tween 20), the clones with binding activity were sorted (n=500) using a flow cytometer (FACSCalibur™, NJ, USA). (4) The selected clones were identified by sequencing the CDR3 region using DNA oligonucleotide sequencing.

### [Determination of Binding Affinity (K_{D}) of Anti-TMPRSS2 Fv]

The K_{D} of the Anti-TMPRSS2 Fv was determined using a SPR biosensor (i-Cluebio, Seongnam, South Korea). The procedure was as follows: (1) The SPR chips, made of BK-7 glass, were coated first with a titanium layer (2 nm), followed by a gold layer (48 nm). (2) Anti-TMPRSS2 Fv (20 μg/mL, 100 μL) was immobilized onto the gold surface of the SPR chops by incubating for 16 h at 4° C. (3) The chips were blocked with bovine serum albumin (BSA, 1 mg/mL) for 1 h at 37° C. (4) After washing with 0.01% PBST, various concentrations of the TMPRSS2 antigen (12.5 to 100.0 nM) were introduced at a flow rate of 25 μL/min for 10 min to facilitate binding. (5) For dissociation, the washing buffer (PBS) was flowed at the same rate for 10 min.

### [Binding Analysis of Anti-TMPRSS2 Fv to TMPRSS2]

The binding interaction between Anti-TMPRSS2 Fv and TMPRSS2 was confirmed using the SPR biosensor analysis as described above. The SARS-COV-2 PPC (20 μg/mL, 100 μL) was immobilized onto the gold surface of the SPR chips for 16 h at 4° C. After washing step with PBS, the binding of TMPRSS2 was monitored before and after treatment with Anti-TMPRSS2 Fv antibodies at varying concentrations (11.1 to 300.0 nM).

### [Production of Pseudo-Virus Particles]

The production of SARS-COV-2 SP variants pseudo-viruses, including strains Wu-1 D614, Delta B.1.617.2, Omicron BA.2, and Omicron BA.4/5, was performed using Lenti-X™ HEK293T cells. The production process comprised the following steps: (1) Lenti-X™ HEK cells (1×10⁵ cells) were cultured in 15 mL of DMEM supplemented with 10% FBS for 1d. (2) A mixture of the transfection reagent FuGENE (10 μL) and 5 μg of each plasmid (SP pseudotyping vectors, pLVXS-ZsGreen1-Puro, and psPAX2) was prepared in 1 mL of Opti-MEM and incubated at 37° C. for 10 min. (3) The cell culture medium was then replaced with 10 mL of fresh DMEM containing 10% FBS, and the transfection mixture from step 2 was added to the cells. (4) After three days of incubation, the supernatant was harvested by centrifugation at 500×g for 10 min. (5) Lenti-X™ concentrator was added to the supernatant and incubated for 3 h at 4° C. (6) The pseudo-virus particles were then pelleted by centrifugation at 1,500×g for 45 min and resuspended in DMEM. (7) The concentration of the pseudo-virus particles was quantified using Lenti-X™ qRT-PCR.

### [Evaluation of Neutralizing Effectiveness of Anti-TMPRSS2 Fv]

The neutralizing effectiveness of the Anti-TMPRSS2 Fv was assessed through an in vitro cell-based infection assay. The procedure was as follows: (1) HEK-Blue™ cells overexpressing hACE2-TMPRSS2 (5.0×10⁴ cells/well) were seeded onto 96-well microplates pre-coated with poly L-lysine and incubated for 1 d. (2) The Anti-TMPRSS2 Fv (20 μg/mL) was added to the HEK-Blue™ cells and incubated for 30 min in 100 μL of DMEM containing 2% FBS at 37° C. (3) Subsequently, the pseudo-virus particles were introduced to the HEK-Blue™ cells, followed by a 48 h incubation period. (4) The cells were Imaged using a fluorescence microscope (Eclipse Ts2) to evaluate the neutralization.

### Results and Discussion

### [Screening of Fv-Antibodies Against TMPRSS2]

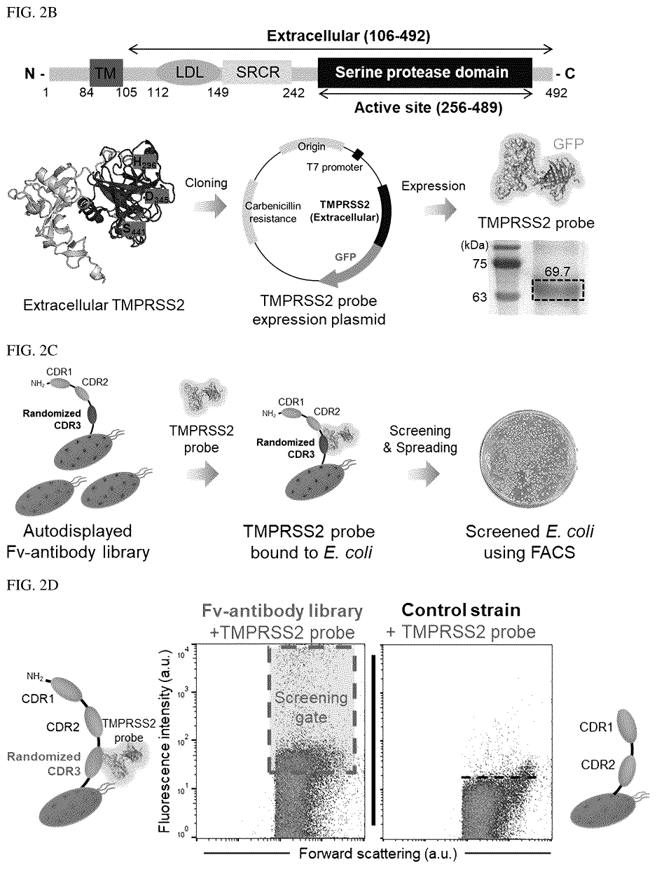

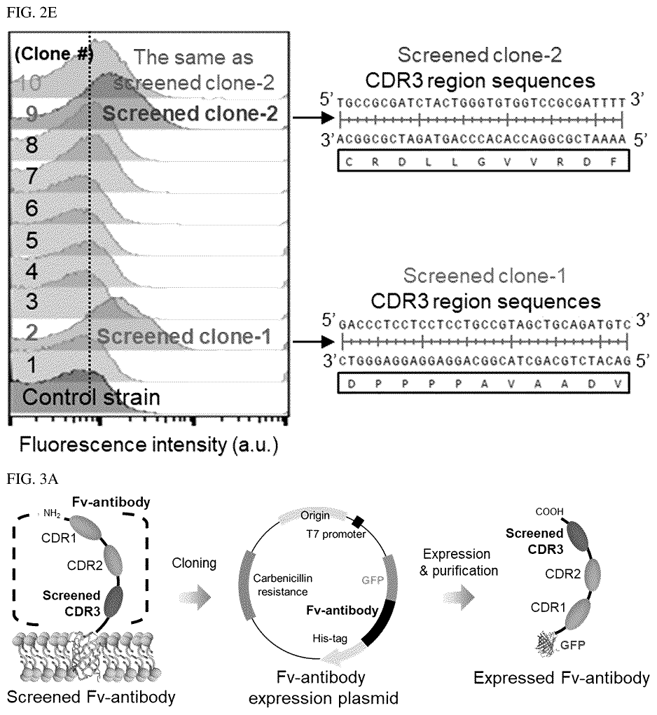

Fv-antibodies with the binding affinity to the active site of TMPRSS2 were screened to prevent the infection of SARS-COV-2 into host cells. As shown in FIG. 2A, Fv-antibody indicated the VH domain of IgG, which consisted of three CDRs and four FRs. The Fv-antibody library was prepared by randomizing the 11 amino acid residues of CDR3 by site-directed mutagenesis. The Fv-antibody library (gene) was expressed on the outer membrane of E. coli by autodisplay technology. So prepared Fv-antibody library on the outer membrane of E. coli had a high surface density of Fv-antibodies upto 10⁵ Fv-antibodies per E. coli and a high diversity of 10⁶ Fv-antibodies per library. Using this Fv-antibody library, TMPRSS2 inhibitors were screened to prevent the infection of SARS-COV-2.

As shown in FIG. 2B, TMPRSS2 is a transmembrane protein with a molecular weight of 53.8 kDa and it is known to be essential for the viral fusion of SARS-COV-2 with the host cell. The extracellular domain of TMPRSS2 (residues: 106-492, PDB No.: 7MEQ) was expressed as a screening probe, which included the active sites (residues: 256-489) and substrate binding site (residues: D435, S460 and G462), and catalytically active site (residues: H296, D345 and S441). This region was located in the pocket where S2′ site of SP was known to specifically bind, facilitating the proteolytic cleavage necessary for viral entry.

As shown in FIG. 2C, the fluorescence-labeled TMPRSS2 probe was reacted to the Fv-antibody library, and positive clones were subsequently screened using flow cytometry. As shown in FIG. 2D, a highly fluorescent region was observed in dot plot of flow cytometry, which indicated the potential binding interactions. In contrast, the control strain with only CDR1 and CDR2 exhibited no significant fluorescence in the same region. These results indicated that the Fv-antibody library contained clones capable of specifically binding to the fluorescent TMPRSS2 probe with the specific binding occurring through the amino acid sequence of CDR3 in the fluorescent clones.

The clones with the fluorescent signals were isolated using flow cytometry and grown on an agar plate. Randomly selected clones were treated with the fluorescent TMPRSS probe, and the binding affinity was analyzed again using flow cytometry. The oligonucleotide sequencing was carried out for the highly fluorescent clones, and two final clones with suitable oligonucleotide sequence in the CDR3 region were finally selected, as shown in FIG. 2E. The oligonucleotide sequences and the corresponding amino acid sequences of screened CDR3 region of Fv-antibodies were summarized in Table 1.

TABLE 1

| | | | Binding |
|---|---|---|---|
| | | | constant |
| Screened | CDR3 region sequence | | (K_{D}) |
| clone | Oligonucleotide (33 bp) | Amino acid (11 mer) | SPR (nM) |
| 1 | 5'-GAC CCT CCT CCT | ¹DPPPP ⁵AVAAD ¹¹V | 36.7 |
| | CCT GCC GTA GCT GCA | | |
| | GAT GTC-3' | | |
| 2 | 5'-TGC CGC GAT CTA | ¹CRDLL ⁵GVVRD ¹¹F | 39.8 |
| | CTG GGT GTG GTC CGC | | |
| | GAT TTT-3' | | |

### [Binding Properties of Screened Fv-Antibodies]

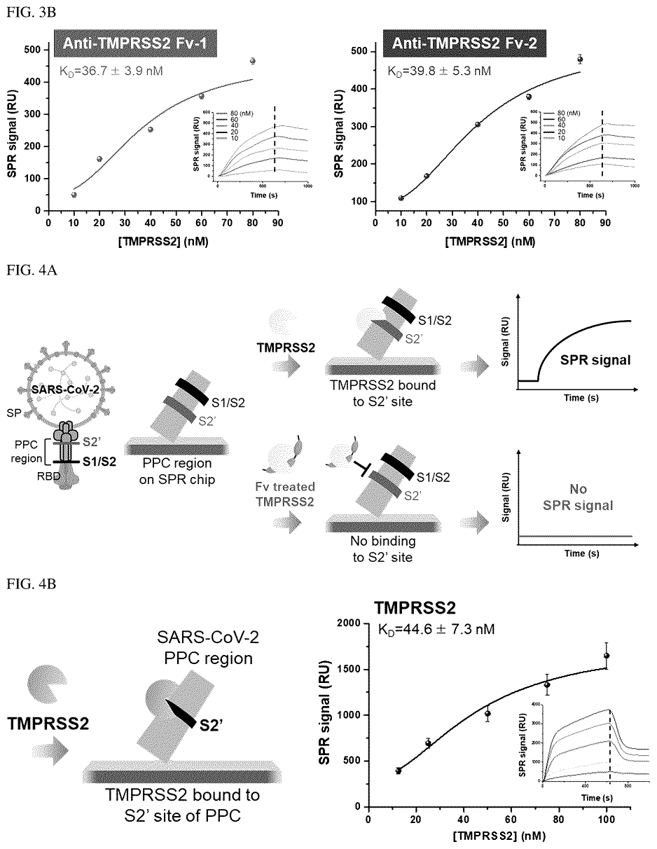

The screened Fv-antibodies were expressed as soluble proteins along with GFP, as shown in FIG. 3A. The co-expression of GFP offered several advantages: (1) it enhanced the solubility of the Fv-antibody, which has limited solubility and (2) it facilitated effective immobilization onto the metal surface of biosensors. The binding affinity of these expressed Fv-antibodies were estimated using SPR biosensor. After these Fv-antibodies were immobilized on Au-chip of SPR biosensor, different concentration of the TMPRSS2 probe was treated and SPR signal was measured. The binding affinity (K_{D}) was calculated to be 36.7 for Anti-TMPRSS2 Fv-1 and 39.8 for Anti-TMPRSS2 Fv-2, as shown in FIG. 3B. These calculated values were comparable to the known binding affinity between TMPRSS2 and the SP of SARS-COV-2 (53 nM), highlighting the potential effectiveness of these Fv-antibodies as inhibitors. The binding affinity (K_{D}) of SARS-COV-1 SP for the host cell ACE2 receptor is reported to range from 31 to 100 nM, while SARS-COV-2 is also reported to have a binding affinity in the range of 4.7 to 10.0 nM.

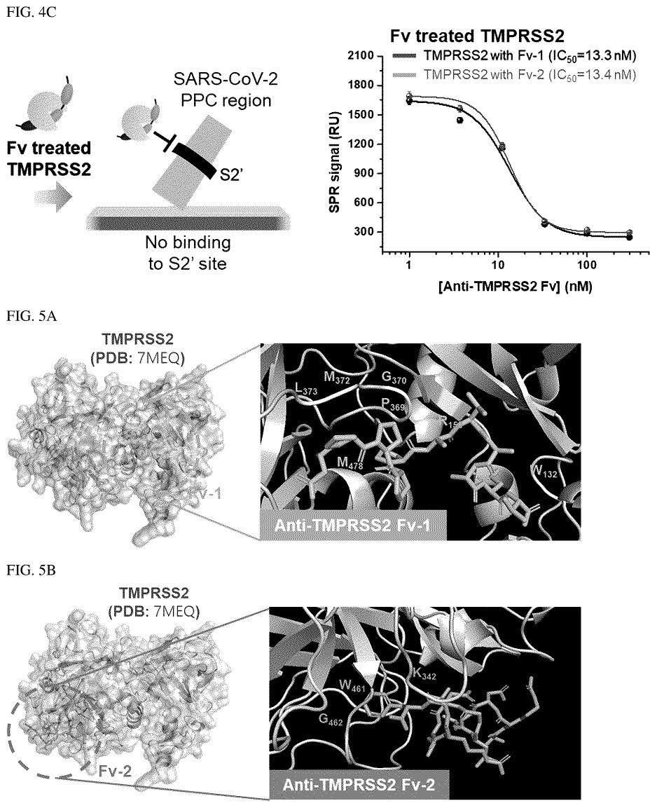

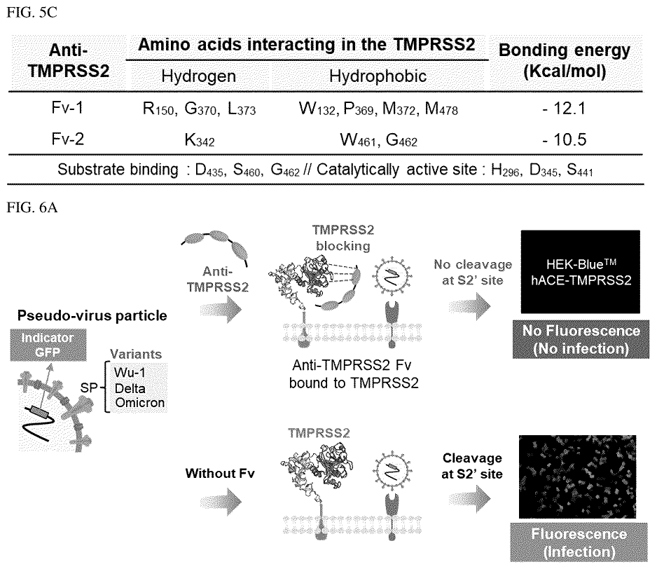

The specific binding of two Fv-antibodies to the active site of TMPRSS2 was confirmed by analyzing the interaction of Fv-antibodies and the PPC region of SARS-COV-2 SP including the S2′ site. The SARS-COV-2 SP contains cleavage sites located in the PPC region, which includes the S1/S2 site (residues: 682-685) targeted by the proteolytic enzyme furin and the S2′ site (residues: 809-815) targeted by TMPRSS2. As shown in FIG. 4A, the PPC region was immobilized on Au-Chip of SPR biosensor, and the binding of TMPRSS2 was monitored before and after treatment with the Fv-antibodies. As shown in FIG. 4B, when the TMPRSS2 probe was treated to the SPR biosensor, there observed a significant signal which indicated the binding of TMPRSS2 to the S2′ site. As shown in FIG. 4C, when TMPRSS2 was pre-incubated with Anti-TMPRSS2 Fv-1, a significantly decreased SPR signal was observed, which indicated blocking of the binding site of Anti-TMPRSS2 Fv-1. A similar result was obtained with the treatment of Anti-TMPRSS2 Fv-2. From these results, the IC50 was determined to be 13.3 nM for Anti-TMPRSS2 Fv-1 and 13.4 nM for Anti-TMPRSS2 Fv-2. Both Fv-antibodies could specifically bind to the active site of TMPRSS2, and it effectively inhibited the proteolytic activity of TMPRSS2.

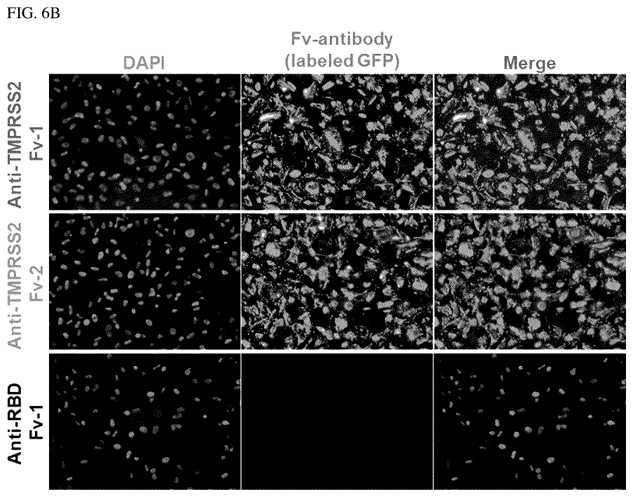

To investigate the binding affinity between TMPRSS2 and the screened Fv-antibodies, docking simulations were carried out using Autodock Vina from Scribbs Research (La Jolla, CA, USA). As shown in FIG. 5A, the interaction of Anti-TMPRSS2 Fv-1 was analyzed to be made at the known active site of TMPRSS2, with the binding energy calculated to be −12.1 kcal/mol, indicating spontaneous binding between TMPRSS2 and Anti-TMPRSS2 Fv-1. The interaction was found to involve hydrogen bonding with R150, G370, L373, and hydrophobic interaction with W132, P369, M372, M478 from Anti-TMPRSS2 Fv-1. As shown in FIG. 5B, the interaction of Anti-TMPRSS2 Fv-2 was analyzed to occur at the active site of TMPRSS2, with a binding energy calculated to be −10.5 kcal/mol, also indicating spontaneous binding. The interaction was found to involve hydrogen bonding with K342 and hydrophobic interaction with W461, G462 from Anti-TMPRSS2 Fv-2. The results were summarized in FIG. 5C, indicating that both Anti-TMPRSS2 Fv-1 and Fv-2, which had high binding affinity, spontaneously bound to the active site (residue: 256-489) of TMPRSS2.

### [Neutralizing Activity of Screened Fv-Antibodies]

The neutralizing activity of Fv-antibodies against SARS-COV-2 infection was evaluated using the cell-based infection assay. The in-vitro cell-based assays employing pseudo-virus particles have been shown to strongly correlate with assays using live SARS-CoV-2 virus. HEK293T cell line stably expressing TMPRSS2 and ACE2 receptor (HEK-Blue™ hACE-TMPRSS2) was utilized as the host cells for SARS-COV-2 infection. Pseudo-virus based on lentivirus were prepared by expressing the SP of SARS-COV-2 corresponding to Wu-1 (D614), Delta (B.1.617.2), Omicron (BA.2), and Omicron (BA.4/5) strains. The pseudo-virus included a GFP expression vector as an infection indicator which produce the fluorescence signal by expression of GFP at the cytosol of host cell after infection.

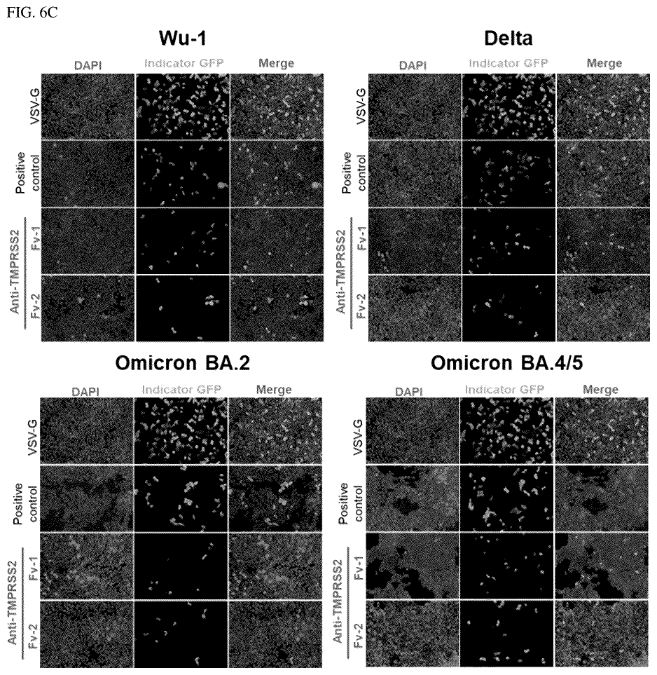

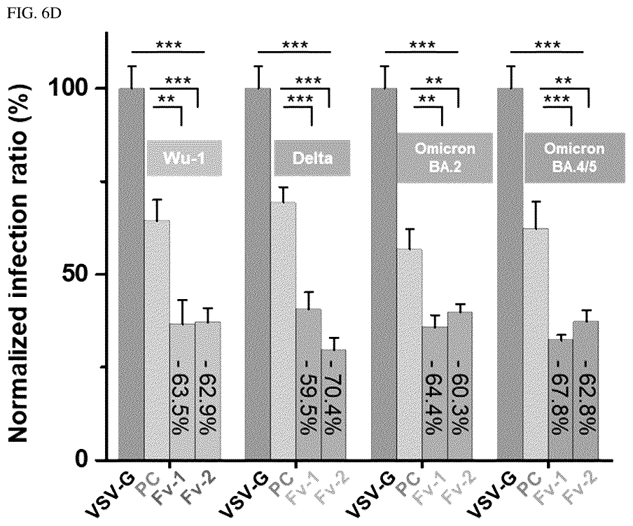

When the pseudo-virus was infected into the host cell, the fluorescence signal was produced according to the expression of GFP as shown in FIG. 6A. When the infection of pseudo-virus was inhibited by Fv-antibodies, there occurred no fluorescence signal. Therefore, the prevention activity of Fv-antibodies from the infection of virus could be estimated through the measurement of fluorescence signal. As the first step, the binding activity of Fv-antibodies (labeled with GFP) to TMPRSS2 on the host cell was observed, as shown in FIG. 6B. In the case of the Fv-antibody against the RBD of SARS-COV-2 SP (labeled with GFP), no specific binding was observed, thus confirming that the binding of two Fv-antibodies was specific to TMPRSS2 on the host cell.

The neutralizing activity of the Fv-antibodies against SARC-CoV-2 infection was then assessed using the pseudo-virus. As shown in FIG. 6C, the infection was carried out using pseudo-virus corresponding to Wu-1 (D614), Delta (B.1.617.2), Omicron (BA.2), and Omicron (BA.4/5) variants. In this regard,, there observed difference in fluorescence signal before and after the treatment of Fv-antibodies. As shown in fluorescence images for four kinds of variant strains of pseudo-virus, the treatment of Fv-antibodies was observed to significantly reduced the fluorescence signals which represented the blockage of virus infection by the Fv-antibodies. For the statistical calculation of neutralizing activity of Fv-antibodies, the fluorescence signal was analyzed using flow cytometry. As shown in FIG. 6D, the neutralizing activity by the Fv-antibodies was estimated to be 59.5 to 70.4% for four kinds of pseudo-virus particles in comparison with the infection of VSV-G. These results represented that (1) the infection of SARS-COV-2 could be effectively prevented by the Fv-antibodies through the inhibition of the protease activity of TMPRSS2 and (2) the neutralizing activity could be achieved to be a similar level for variant strains of SARS-COV-2.

### CONCLUSIONS

In the present disclosure, Fv-antibodies against TMPRSS2 were screened from the Fv-antibody library. This Fv-antibody library (gene) was prepared by site-directed mutagenesis of the CDR3 region, and the Fv-antibody library was expressed on the outer membrane of E. coli using autodisplay technology which had an expression level exceeding 10⁵ Fv-antibodies per E. coli and a diversity of over 10⁶ Fv-antibodies per library. After the screening process, the selected Fv-antibodies were expressed as soluble recombinant proteins. Using the SPR biosensor the binding affinities (K_{D}) of these antibodies were measured for Anti-TMPRSS2 Fv-1 and Fv-2 to be 36.7 nM and 39.8 nM, respectively. Additionally, the IC50 was determined to be 13.3 nM for Anti-TMPRSS2 Fv-1 and 13.4 nM for Anti-TMPRSS2 Fv-2. The docking simulation demonstrated that these Fv-antibodies specifically bind to the active sites of TMPRSS2, thereby effectively blocking these regions of the protease.

The neutralizing activity of the Anti-TMPRSS2 Fv-antibodies was tested using an in vitro cell-based infection assay. In this assay, pseudo-viruses with the expressed SP of SARS-COV-2 variants were used for the assay, such as Wu-1 (D614), Delta (B.1.617.2), Omicron (BA.2), and Omicron (BA.4/5). The results showed that the neutralizing activity of the Fv-antibodies ranged from −59.5% to −70.4%. These results showed that the screened Anti-TMPRSS2 Fv-antibodies were capable of effectively inhibiting TMPRSS2 through binding to its activity sites, and the neutralizing activity of Fv-antibodies against TMPRSS2 could be obtained for SARS-COV-2 variants, such as Wu-1 (D614), Delta (B.1.617.2), Omicron (BA.2), and Omicron (BA.4/5).

Table 2 is a table showing information regarding the CDR sequences of the peptides and antibodies used in the present disclosure.

TABLE 2

| Sequence | | | | |
|---|---|---|---|---|
| ID | Sequence | Molecule | | |
| Number | Name | Type | Organism | Sequence |
| 1 | peptide 1 | AA | Synthetic | DPPPPAVAADV |
| | | | construct | |
| 2 | peptide_2 | AA | Synthetic | CRDLLGVVRDF |
| | | | construct | |
| 3 | CDR1 | AA | synthetic | TYGIQ |
| | | | construct | |
| 4 | CDR2 | AA | synthetic | WIHAGTGGTK |
| | | | construct | YSRKFQG |

Although the embodiments of the present disclosure have been described above with reference to the accompanying drawings, the present disclosure may not be limited to the embodiments and may be implemented in various different forms. Those of ordinary skill in the technical field to which the present disclosure belongs will be able to appreciate that the present disclosure may be implemented in other specific forms without changing the technical idea or essential features of the present disclosure. Therefore, it should be understood that the embodiments as described above are not restrictive but illustrative in all respects.

What is claimed is:

1. A peptide capable of specifically binding to a cleavage site of SARS-COV-2 spike protein by TMPRSS2 (transmembrane protease serine 2), wherein the peptide comprises a peptide sequence of SEQ ID NO: 1 or a peptide sequence of SEQ ID NO: 2:
[SEQ ID NO: 1]
DPPPPAVAADV
[SEQ ID NO: 2]
CRDLLGVVRDF.

2. The peptide of claim 1, wherein the peptide binds to a S2′ site of the SARS-COV-2 spike protein to block infection thereof into a host cell.

3. A nucleic acid coding the peptide according to claim 1.

4. A recombinant expression vector including the nucleic acid according to claim 3.

5. A cell transformed with the recombinant expression vector according to claim 4.

6. An antibody or antigen-binding fragment thereof capable of specifically binding to a cleavage site of SARS-COV-2 spike protein by TMPRSS2,
wherein a CDR3 site of the antibody or antigen-binding fragment thereof comprises a peptide sequence of SEQ ID NO: 1 or a peptide sequence of SEQ ID NO: 2:
[SEQ ID NO: 1]
DPPPPAVAADV
[SEQ ID NO: 2]
CRDLLGVVRDF.

7. The antibody or antigen-binding fragment thereof claim 6, wherein the CDR3 site of the antibody or antigen-binding fragment thereof binds to a S2′ site of the SARS-COV-2 spike protein to block infection thereof into a host cell.

8. A composition for preventing SARS-COV-2 infection, the composition comprising the antibody or antigen-binding fragment thereof according to claim 7.
